(19) United States
(12) Patent Application Publication
(10) Pub. No.: US 2010/0255044 A1
(43) Pub. Date: Oct. 7, 2010

(54) METHOD OF DEPOSITING PARTICULATE BENEFIT AGENTS ON KERATIN-CONTAINING SUBSTRATES

(76) Inventors: SUSAN DALY, BASKING RIDGE, NJ (US); JANUSZ JACHOWICZ, BETHEL, CT (US); ROBERT BIANCHINI, HILLSBOROUGH, NJ (US); ALLWYN COLACO, SOUTH RIVER, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/722,143

(22) Filed: Mar. 11, 2010

Related U.S. Application Data
(60) Provisional application No. 61/164,676, filed on Mar. 30, 2009.

(51) Int. Cl. A61K 8/64; A61K 8/02; A61Q 5/10; A61Q 11/00; A61Q 3/00; A61Q 1/02; B05D 1/36

(52) U.S. Cl. 424/401; 424/70.6; 424/61; 427/414; 977/926

(57) ABSTRACT

This invention relates to the deposition of particulate benefit agents onto keratin-containing substrates such that the deposited particulates are resistant to removal by exposure to surfactants or cleansing agents. The deposition is accomplished by sequentially treating the substrate with a cationic composition followed by treatment with an anionic particulate benefit agent.

This patent application claims the benefit of priority of U.S. Provisional Patent Application Ser. No. 61/164,676.

### FIELD OF THE INVENTION

This invention relates to methods and kits for depositing particulate benefit agents, including colored pigments, microparticles, nanoparticles, and other beneficial particulates onto keratin-containing substrates such as hair, skin, nails, and wool, such that the deposited pigments or particulates are resistant to removal by exposure to surfactants or cleansing agents. The method involves a deposition of a first cationic compound onto the keratin-containing substrate to form a layer, followed by the deposition of anionic particles, which may include colored pigments, nanoparticles, microparticles or proteins onto this layer.

### BACKGROUND OF THE INVENTION

For many years, individuals with graying or fading hair colors have sought the use of permanent or semi-permanent dyes to change the color of their hair. Such dyes generally operate under oxidative and direct dyeing processes and cause substantial damage to the hair of individuals utilizing such dyes. For example, in order to permit the dye to penetrate into the hair to effect a color change, the individual must cause the cuticle of the hair to open, usually by oxidizing the hair. This process causes great damage to the hair cuticle, drying the hair. This can result in hair breakage and lend an unhealthy appearance and texture to the hair. Despite these disadvantages, however, this process has come to be the most acceptable means of achieving desired shades and colors in hair.

Pigment, defined as a “fine insoluble white, black, or colored material” [Julius Grant, editor, Hackh's Chemical Dictionary, McGraw-Hill Book Company, New York, 1969], has also been utilized to change the color of substrates. However, pigments are rarely used in hair coloring due to the complexities of applying and retaining the pigment on hair. The geometry of pigment particles, their size, index of refraction, and surface properties are among the characteristics of pigments that make them difficult to use in hair coloring processes. Because most pigments or colored particles are anionic in charge, they do not deposit readily onto anionically charged surfaces, such as keratin-containing substrates.

Although pigments are used in mascara, they are present in these compositions in very high loadings, on the order of 10 to 30 weight percent. Furthermore, pigments must be held in place using film-forming polymers or other styling polymers. These compositions must coat the hair and adhere the pigments to the hair surface. These mechanical bonds are fairly loose and thus such pigment compositions are quite easy to remove. Coating longer hairs with polymers may also cause the hairs' texture and appearance to become unnatural and result in difficulty in managing the hair. Thus, utilizing pigment particles in compositions intended to color hair has been problematic and undesirable.

In addition, pigments are difficult to use in cosmetic applications that require detergents, conditioning agents, thickeners, silicones, solvents, inorganic and organic salts, humectants and other typical cosmetic ingredients. This is due to pigments' tendency to deposit competitively on hair substrates, causing other hair benefit agents to fail to deposit and bond with the substrates. Furthermore, pigments tend to be incompatible with such materials in formulations. Thus, formulations containing pigments may lack several desirable consumer benefits.

Moreover, due to their insoluble nature, pigment-containing formulations are generally difficult to stabilize. The pigments must be suspended in the compositions so as not to precipitate in an esthetically undesirable manner.

The deposition of other particulate materials onto hair or other keratin-containing substrates could also provide benefits to those substrates. Such beneficial particles include sunscreen particles, dye-doped particles, sparkling particles, and microspheres containing conditioning agents, antimicrobial agents, antifungal agents, fragrances, anti-lyses agents, aromatherapy agents, insect repellents, and the like. However, the deposition and adherence of these types of particles present the same challenges as those encountered for the deposition of pigment particles.

Thus, heretofore, there has not been means for depositing other beneficial particles onto keratin-containing substrates such that the particles are resistant to being easily washed off or removed. Likewise, there has not been means for affecting keratin fiber color by sustainably attaching pigments to the keratin fibers such that the color is resistant to being washed off or otherwise easily removed.

### SUMMARY OF THE INVENTION

Surprisingly, it has been found that, by first depositing a layer of a cationic material onto a keratin-containing substrate, a particulate benefit agent may then be deposited onto the keratin-containing substrate such that the benefit agent remains even after subsequent exposure to fluids, including surfactants or other cleansing agents and methods.

The compositions and methods of this invention relate to compositions, methods and kits for depositing a particulate benefit agent onto a keratin-containing substrate by sequentially:

a) providing a first cosmetic composition having at least one cationic compound selected from cationic proteins, cationic peptides, cationic polymers, or mixtures of these;

b) applying said first cosmetic composition to a keratin-containing substrate for a time period sufficient for at least one cationic compound to be deposited on the substrate and form a layer;

c) optionally, rinsing the first cosmetic composition from the substrate with water;

d) providing a second cosmetic composition having at least one anionic particulate benefit agent;

e) applying the second cosmetic composition to the keratin-containing substrate for a time period sufficient for the at least one anionic particulate benefit agent to be deposited on the layer; and

f) optionally, rinsing the second cosmetic composition from the substrate with water.

The compositions and methods of this invention also relate to compositions, methods and kits for coloring a keratin-containing substrate by sequentially:

a) providing a first cosmetic composition having at least one cationic compound selected from cationic proteins, cationic peptides, cationic polymers, or mixtures of these;

b) applying said first cosmetic composition to a keratin-containing substrate for a time period sufficient for at least one cationic compound to be deposited on the substrate and form a layer;

c) optionally rinsing the first cosmetic composition from the substrate with water;

d) providing a second cosmetic composition having at least one anionic colored pigment, microparticle, or nanoparticle;

e) applying the second cosmetic composition to the keratin-containing substrate for a time period sufficient for the at least one colored pigment, microparticle, or nanoparticle to be deposited on the layer; and

f) optionally rinsing the second cosmetic composition from the substrate with water.

Unexpectedly, we have also found that the layer deposited on the keratin-containing substrate is durable under cleansing treatments. Thus, the keratin-containing substrate may be cleansed with a composition containing a surfactant or other cleansing agent after the first cosmetic composition is applied to it to form the layer. Surprisingly, after such a cleansing, there is still good deposition of the particulate or pigment onto the layer when the second cosmetic composition is applied.

The particulate or pigment of the second cosmetic composition may be coated or uncoated. Coated pigments or particulates may be anionic, hydrophilic, or hydrophobic. Anionically coated pigments and particulates may be used without further dispersants in the second cosmetic composition, as their anionic coatings provide them with a negative “zeta potential”, as defined below. Uncoated or hydrophilically or hydrophobically coated pigments or particulates may be rendered anionic, i.e., having a negative zeta potential, by the addition of an anionic dispersant to the second cosmetic composition.

Sequential application of the first and second cosmetic compositions of the invention may be repeated one or more times to deposit additional layers onto the keratin-containing substrate. Additional layers of pigment provide enhanced color intensity and improved resistance to wash-out (i.e., leaching and disengagement of pigment, microparticle or nanoparticles via exposure to cleansing products) by surfactant treatment or cleansing. Likewise, additional layers of particulate benefit agent provide enhanced benefits to the keratin-containing substrate and improved resistance to wash-out by surfactant treatment or cleansing.

Other features and advantages of this invention will be apparent from the detailed description of the invention and from the claims.

### BRIEF DESCRIPTION OF THE DRAWINGS

FIG. 1 shows photographs of wool swatches treated according to the methods described in Example 1.

### DETAILED DESCRIPTION OF THE INVENTION

### Definitions

“Keratin-containing substrate”, as used herein, includes hair, skin, nails, teeth, tissues, wool, fur, and any other materials that contain keratin proteins. The keratin-containing substrate useful in the methods of this invention is preferably human hair, skin, or nail.

“Cationic”, as used herein, is used to describe a compound or material with a positive charge. Such compounds generally move toward the negative electrode in electrolysis.

“Anionic”, as used herein, is used to describe a compound or material with a negative charge. Such compounds generally move toward the positive electrode in electrolysis.

“Peptide”, as used herein, is a molecule containing two or more amino acids joined by a peptide bond or modified peptide bonds.

The term “amino acid” refers to the basic chemical structural unit of a protein or polypeptide. The following abbreviations are used herein to identify specific amino acids:

TABLE 1

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Protein”, as used herein, relates to a long chain of amino acids joined together by peptide bonds.

“Polymer”, as used herein, relates to a large organic molecule formed by combining many smaller molecules (monomers) in a regular pattern.

“Isoelectric Point” or “IEP” or “pI”, as used herein, refers to the pH value at which a substance, compound, molecule, or surface carries no net electrical charge or shows no migration under the influence of an electric field.

“Zeta potential”, as used herein, relates to an electrokinetic measurement in a colloidal system. Zeta potential is the average electrical potential in the hydrodynamic plane of shear, separating the bulk liquid phase and the diffuse layers of the electrochemical double layer, and can be calculated from the streaming potential or streaming current measurement. (Reference: Instruction Manual for SurPass Electrokinetic Analyzer, Anton Paar, GmbH, Document No. A481B30-A, Austria, 2006.)

“Particle” or “particulate”, as used herein, refers to a small, discrete portion of material that has mass and dimension. For purposes of this invention, particles include microparticles and nanoparticles.

“Microparticle”, as used herein, refers to a particle having a diameter ranging from about 1 to about 1000 micrometers.

“Nanoparticle”, as used herein, refers to a particle having a diameter ranging from about 1 to about 1000 nanometers.

“Pigment”, as used herein, refers to a fine, insoluble white, black or colored material. For the purposes of this invention, pigments also include pigment microparticles and pigment nanoparticles.

“Diameter”, as used herein, refers to the largest side-to-side linear dimension of a particle, microparticle, or nanoparticle.

The phrase “stable dispersion” as used herein, refers to_a dispersion or suspension of one phase (solid or liquid) in another phase (solid or liquid) in which the dispersed particles do not undergo coagulation, precipitation, and/or phase separation on standing at ambient conditions for an extended period of time.

The compositions, methods and kits of this invention provide coloring or other benefits to keratin-containing substrates by the sequential deposition of a cationically charged layer onto the keratin-containing substrate followed by the deposition of an anionically charged particulate benefit agent or colored pigment or particulate onto the substrate. Unexpectedly, the benefit or color provided to the keratin-containing substrate is retained even after cleansing and exposure to surfactants. Thus, the method of the invention may be used for depositing particulate benefit agents or colored pigments or particles onto hair such that the benefit agent or color does not wash out after shampooing.

The initial step of the method of the invention involves providing a first cosmetic composition containing a net positively charged (i.e., cationic) compound and applying this first composition to a keratin-containing substrate, such as, for example, human hair, to form a first layer. The cationic compound deposits onto the anionically charged surface of the hair, thus reversing its zeta potential from negative to positive or, at least, reducing the negative potential of the hair surface to low values.

The next step of the invention involves providing a second cosmetic composition containing an anionically charged particulate benefit agent or colored pigment, microparticle, or nanoparticle, and sequentially applying this second composition to the hair. The particulate benefit agent or colored pigment or particulate in the second composition is anionically charged by virtue of either: a) an anionic coating, or b) an anionic dispersant in the composition. In either case, the application of the second composition results in the deposition of a net negatively charged layer of particulate benefit agents or colored pigment onto the earlier deposited positively charged layer. The preferred second layer functions to return the zeta potential of the hair to a neutral or slightly negative charge.

It is believed that one skilled in the art can, based upon the description herein, utilize the compositions and methods of this invention to their fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### Cationic Compounds

Cationic compounds useful in the compositions and methods of this invention include cationic proteins, cationic peptides, cationic polymers, and mixtures of these.

Cationic proteins include naturally-occurring cationic proteins and synthetic cationic proteins. Examples of naturally-occurring cationic proteins include lysozyme; avidin; methylated collagen; Cytochrome C; Platelet Factor 4; Protamine sulfate; Telomerase; cationic proteases, including trypsin, chymotrypsin, papain, caspase; RNA or DNA binding proteins, including histones, Ribonuclease A, and Deoxyribonuclease (DNase); and antimicrobial proteins, including magainin, defensins, and cathelicdin. Examples of cationic synthetic proteins include polylysine, polyarginine, polyhistidine, copolymers of these, or proteins containing a molar fraction of 50% or more of lysine, arginine, or histidine amino acids. Examples include poly (Glu, lys) hydrobromide, poly (Lys, Tyr) hydrobromide, poly (Ala, Glu, Lys, Tyr) hydrobromide, and poly (Arg, Trp) hydrobromide all available from Sigma Aldrich.

Cationic peptides include, for example, polylysine, polyarginine, polyhistidine, or copolymers or peptides containing a greater total number of basic amino acids than acidic amino acids. In other words, these copolymers or peptides will have a net electrical charge of at least 1 at neutral pH (about 6 to about 7.5). Examples of basic amino acids include lysine, arginine, and histidine. Examples of acidic amino acids include aspartic acid and glutamic acid.

Cationic polymers include naturally-occurring cationic polymers and synthetic cationic polymers. Examples of naturally-occurring cationic polymers include, without limitation, chitosan, polyquaternium-4, polyquaternium-10, polyquaternium-24, and modifications of these. Examples of synthetic cationic polymers include, without limitation, synthetic cationic polymers with one or more primary amines, synthetic cationic polymers with one or more secondary amines, synthetic cationic polymers with one or more tertiary amines, synthetic cationic polymers with one or more quaternary amines, and mixtures of these. Specific examples of synthetic cationic polymers include, without limitation, homopolymers or copolymers derived from acrylic or methacrylic esters or amides, such as poly methacrylamidopropyltrimethylammonium chloride, polyquaternium-1, polyquaternium-2, polyquaternium-5, polyquaternium-6, polyquatenium-7, polyquaternium-8, polyquaternium-11, polyquaternium-16, polyquaternium-17, polyquaternium-18, polyquaternium-22, polyquaternium-27, polyquaternium-28, polyquaternium 31, polyquaternium-39, polyquaternium-43, polyquaternium-44, polyquaternium-46, polyquaaternium-47, polyquaternium-53, polyquaternium-55, PVP/dimethylaminoethyl methacrylate copolymer, VP/dimethylaminoethyl methacrylate copolymer, VP/DMAPA acrylate copolymer, VP/vinyl caprolactam/DMAPA acrylates copolymer, vinylcaprolactam/PVP/dimethylaminoethylmethacrylate copolymer, and mixtures of these and the like.

The cationic compounds of this invention preferably have an Isoelectric Point of about 8 to about 12.

The cationic compounds used in this invention have a concentration range in the compositions of the first cosmetic composition of this invention of from about 0.000001% to about 10% by weight, more preferably from about 0.001% to about 5% by weight, and even more preferably from about 0.01% to about 2% by weight.

### Particulate Benefit Agents

The particulate benefit agents contained in the second cosmetic composition of this invention may be particles, including microparticles or nanoparticles, containing compositions or agents with properties that impart benefits to a keratin-containing substrate when deposited thereon. Nonlimiting examples of such particulate benefit agents include sunscreen agents, antimicrobial agents, sparkling particles, odor-control agents, and microspheres containing conditioning agents, anti-fungal agents, fragrances, anti-lyses agents, aromatherapy agents, insect repellent agents, and the like. Nonlimiting examples of particulate sunscreen agents include inorganic particulates, such as zinc oxide and titanium dioxide; and organic particulates, such as methylene bis-benzotriazolyl tetramethylbutylphenol (available as Bisoctrizole from Ciba Specialty Chemicals of Basel, Switzerland). Nonlimiting examples of particulate antimicrobial agents include silver-based particles and activated carbon-based particles. Examples of microspheres containing particulate benefit agents may include encapsulated or microencapsulated benefit agents, which retain the benefit agent within the encapsulation during application and allow the benefit agent to be released from the encapsulation at some desired time after deposition on the keratin-containing surface. Examples of odor-control agents include activated carbon particles and zeolites.

The particulate benefit agents contained in the second cosmetic composition of this invention may also be colored particulates, including colored pigments, colored particles, such as microparticles or nanoparticles, or combinations of these.

Pigments, particularly metal compounds or semimetallic compounds, may be used in the compositions and methods of this invention in ionic, nonionic or oxidized form. The pigments may be in this form either individually or in admixture or as individual mixed oxides or mixtures thereof, including mixtures of mixed oxides and pure oxides. Examples are the titanium oxides (for example TiO₂), zinc oxides (for example ZnO), aluminum oxides (for example Al₂O₃), iron oxides (for example Fe₂O₃), manganese oxides (for example MnO), silicon oxides (for example SiO₂), silicates, cerium oxide, zirconium oxides (for example ZrO₂), barium sulfate (BaSO₄) or mixtures thereof and the like. Suitable pigments are commercially available. An example is Hombitec® L5 (INCI name: titanium dioxides) supplied by Merck.

Other examples of pigments include the following: D&C Red No. 36, D&C Red No. 30, D&C Orange No. 17, Green 3 Lake, Ext. Yellow 7 Lake, Orange 4 Lake, Red 28 Lake, the calcium lakes of D&C Red Nos. 7, 11, 31 and 34, the barium lake of D&C Red No. 12, the strontium lake D&C Red No. 13, the aluminum lakes of FD&C Yellow No. 5 and No. 6, the aluminum lakes of FD&C No. 40, the aluminum lakes of D&C Red Nos. 21, 22, 27, and 28, the aluminum lakes of FD&C Blue No. 1, the aluminum lakes of D&C Orange No. 5, the aluminum lakes of D&C Yellow No. 10; the zirconium lake of D&C Red No. 33, CROMOPHTHAL® Yellow, SUNFAST® Magenta, SUNFAST® Blue, iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, titanium dioxide nanoparticles, zinc oxide, barium oxide, ultramarine blue, bismuth citrate, hydroxyapatite, zirconium silicate, carbon black particles and the like.

The pigments or particles of this invention can be coated or uncoated, and coated particles can be anionic, hydrophilic, or hydrophobic. Suitable anionic coatings include, for example, silica, aluminosilicate, sodium C14-16 olefin sulfonate, disodium stearoyl glutamate, sodium stearoyl glutamate/sodium trideceth-6 carboxylate, and sodium polyacrylates/hydrogenated lecithin/aluminum hydroxide. Examples of uncoated pigments suitable for use in the present invention are given in Table 2.

TABLE 2
Uncoated Pigments

| Pigment Name | Chemical Name | Surface Coating | Source |
|---|---|---|---|
| SOFT TEX BLACK | Iron Oxide | Uncoated | Sun |
| | | | Chemical |
| | | | Corporation |
| | | | Cincinnati |
| | | | OH |
| SUNCROMA RED | Iron Oxide | Uncoated needle | Sun |
| | | shape | Chemical |
| | | | Corporation |
| | | | Cincinnati |
| | | | OH |
| SOFT TEX RED | Iron Oxide | Uncoated | Sun |
| | | | Chemical |
| | | | Corporation |
| | | | Cincinnati |
| | | | OH |
| NANOPHASE NANOARC COSMETIC | Iron Oxide | Uncoated | Nanophase |
| IRON OXIDE (RED) | | | Technologies |
| | | | Corporation, |
| | | | Romeoville, |
| | | | IL |
| NANOPHASE NANOARC BLACK IRON | Iron Oxide | Uncoated | Nanophase |
| OXIDE | | | Technologies |
| | | | Corporation, |
| | | | Romeoville, |
| | | | IL |
| SENSIENT UNIPURE RED LC381 | Iron Oxide | Uncoated | LCW - |
| EM | | | Sensient |
| | | | Cosmetic |
| | | | Technologies |
| | | | Saint Ouen |
| | | | L'Aumone, |
| | | | France |
| SENSIENT UNIPURE BLACK LC989 | Iron Oxide | Uncoated | LCW - |
| | | | Sensient |
| | | | Cosmetic |
| | | | Technologies |
| | | | Saint Ouen |
| | | | L'Aumone, |
| | | | France |
| SENSIENT UNIPURE YELLOW LC182 | Iron Oxide | Uncoated | LCW - |
| EM | | | Sensient |
| | | | Cosmetic |
| | | | Technologies |
| | | | Saint Ouen |
| | | | L'Aumone, |
| | | | France |
| SENSIENT UNIPURE RED LC383 | Iron Oxide | Uncoated | LCW - |
| EM | | | Sensient |
| | | | Cosmetic |
| | | | Technologies |
| | | | Saint Ouen |
| | | | L'Aumone, |
| | | | France |
| SENSIENT UNIPURE RED LC386 | Iron Oxide | Uncoated | LCW - |
| EM | | | Sensient |
| | | | Cosmetic |
| | | | Technologies |
| | | | Saint Ouen |
| | | | L'Aumone, |
| | | | France |
| SENSIENT UNIPURE WHITE LC981 | Titanium Dioxide | Uncoated | LCW - |
| EM | | | Sensient |
| | | | Cosmetic |
| | | | Technologies |
| | | | Saint Ouen |
| | | | L'Aumone, |
| | | | France |
| SENSIENT UNIPURE YELLOW LC181 | Iron Oxide | Uncoated | LCW - |
| | | | Sensient |
| | | | Cosmetic |
| | | | Technologies |
| | | | Saint Ouen |
| | | | L'Aumone, |
| | | | France |
| SENSIENT UNIPURE YELLOW LC380 | Iron Oxide | Uncoated | LCW - |
| | | | Sensient |
| | | | Cosmetic |
| | | | Technologies |
| | | | Saint Ouen |
| | | | L'Aumone, |
| | | | France |
| SENSIENT UNIPURE BROWN LC881 | Iron Oxide | Uncoated | LCW - |
| | | | Sensient |
| | | | Cosmetic |
| | | | Technologies |
| | | | Saint Ouen |
| | | | L'Aumone, |
| | | | France |
| SENSIENT UNIPURE BROWN LC887 | Iron Oxide | Uncoated | LCW - |
| | | | Sensient |
| | | | Cosmetic |
| | | | Technologies |
| | | | Saint Ouen |
| | | | L'Aumone, |
| | | | France |
| SENSIENT UNIPURE BROWN LC889 | Iron Oxide | Uncoated | LCW - |
| | | | Sensient |
| | | | Cosmetic |
| | | | Technologies |
| | | | Saint Ouen |
| | | | L'Aumone, |
| | | | France |
| KOBO TAROX IROX TRR-100 | Iron Oxide | Uncoated | Kobo |
| | | | Products, |
| | | | Inc. South |
| | | | Plainfield |
| | | | NJ |
| KOBO BLACK NF | Iron Oxide | Uncoated | Kobo |
| | | | Products, |
| | | | Inc. South |
| | | | Plainfield |
| | | | NJ |

Examples of anionic coated pigments are given in Table 3.

TABLE 3
Anionic Coated Pigments

| Pigment Name | Chemical Name | Surface Coating | Source |
|---|---|---|---|
| SYMPHOLIGHT RW | Iron Oxide | silica treated | Presperse, |
| | | | Inc. |
| | | | Somerset, |
| | | | NJ |
| SYMPHOLIGHT BW | Iron Oxide | silica treated | Presperse, |
| | | | Inc. |
| | | | Somerset, |
| | | | NJ |
| SP-4405 Surface Passivated | Iron Oxide | aluminosilicate | Color |
| Black Oxide | | | Techniques, |
| | | | Inc., South |
| | | | Plainfield |
| | | | NJ |
| SP-Surface Passivated | Titanium Dioxide | aluminosilicate | Color |
| titanium dioxide | | | Techniques, |
| | | | Inc., South |
| | | | Plainfield |
| | | | NJ |
| SINERT BP-10 | Iron Oxide | silica | Kobo |
| | | | Products, |
| | | | Inc. South |
| | | | Plainfield |
| | | | NJ |
| SINERT RP5-10 | Iron Oxide | silica | Kobo |
| | | | Products, |
| | | | Inc. South |
| | | | Plainfield |
| | | | NJ |
| BRO-C5 | Iron Oxide | silica | Kobo |
| | | | Products, |
| | | | Inc. South |
| | | | Plainfield |
| | | | NJ |
| AQUASPERSABIL RIO | Iron Oxide | sodium C₁₄₋₁₆ Olefin | Presperse, |
| | | Sulfonate | Inc. |
| | | | Somerset, |
| | | | NJ |
| AMINO ACID (NAID) TREATED | Iron Oxide | Disodium Stearoyl | U.S. |
| IRON OXIDE | | Glutamate | Cosmetics |
| | | | Corporation, |
| | | | Dayville |
| | | | CT |
| BLACK BL-100 SPA | Iron Oxide | Sodium Stearoyl | Kobo |
| | | Glutamate (And) | Products, |
| | | Sodium Trideceth-6 | Inc. South |
| | | Carboxylate | Plainfield |
| | | | NJ |
| PALI TREATED IRON OXIDE | Iron Oxide | Sodium | U.S. |
| | | Polyacrylates/Hydrogenated | Cosmetics |
| | | Lecithin/ | Corporation, |
| | | Aluminum Hydroxide | Dayville |
| | | | CT |

Examples of hydrophilic coated pigments are given in Table 4.

TABLE 4
Hydrophilic Coated Pigments

| | Chemical | | |
|---|---|---|---|
| Pigment Name | Name | Surface Coating | Source |
| CT-2 Black BL-100P | Iron Oxide | chitosan | Kobo |
| | | | Products, |
| | | | Inc. South |
| | | | Plainfield |
| | | | NJ |
| CT-2 Red R--516P | Iron Oxide | chitosan | Kobo |
| | | | Products, |
| | | | Inc. South |
| | | | Plainfield |
| | | | NJ |
| BGRO-SW2 | Iron Oxide | PEG-8 Methyl | Kobo |
| | | Ether | Products, |
| | | Triethoxysilane | |
| | | | Inc. South |
| | | | Plainfield |
| | | | NJ |
| GA-7403 Galactoarabinan | Iron Oxide | Galactoarabinan | Color |
| treated black oxide | | | Techniques, |
| | | | Inc., South |
| | | | Plainfield |
| | | | NJ |
| Lauroyl Lysine treated iron | Iron Oxide | Lauroyl Lysine | Color |
| oxide | | | Techniques, |
| | | | Inc., South |
| | | | Plainfield |
| | | | NJ |
| C2-5 Black BL-100 | Iron Oxide | cellulose | Kobo |
| | | | Products, |
| | | | Inc. South |
| | | | Plainfield |
| | | | NJ |
| C2-5 Red R-516L | Iron Oxide | cellulose | Kobo |
| | | | Products, |
| | | | Inc. South |
| | | | Plainfield |
| | | | NJ |

Examples of hydrophobic coated pigments are given in Table 5.

TABLE 5
Hydrophobic Coated Pigments

| Pigment Name | Chemical Name | Surface Coating | Source |
|---|---|---|---|
| BBO-I2 | Iron Oxide | Isopropyl Titanium | Kobo |
| | | Triisostearate | Products, |
| | | | Inc. South |
| | | | Plainfield |
| | | | NJ |
| ASC-2 Black BL-100 | Iron Oxide | Acrylates/Dimethicone | Kobo |
| | | Copolymer | Products, |
| | | | Inc. South |
| | | | Plainfield |
| | | | NJ |
| BBO/MM1.5 | Iron Oxide | Magnesium | Kobo |
| | | Myristate | Products, |
| | | | Inc. South |
| | | | Plainfield |
| | | | NJ |
| BBO-11S2 | Iron Oxide | Triethoxy | Kobo |
| | | Caprylylsilane | Products, |
| | | | Inc. South |
| | | | Plainfield |
| | | | NJ |
| ASC-2 BLACK BL-100 | Iron Oxide | Acrylates/Dimethicone | Kobo |
| | | Copolymer | Products, |
| | | | Inc. South |
| | | | Plainfield |
| | | | NJ |
| PF-5 BLACK BL-100 | Iron Oxide | C9-15 | Kobo |
| | | Fluoroalcohol | Products, |
| | | Phosphates | Inc. South |
| | | | Plainfield |
| | | | NJ |
| PF-10 STT-65C-S | titanium dioxide | C9-15 | Kobo |
| | | Fluoroalcohol | Products, |
| | | Phosphates (And) | Inc. South |
| | | Triethanolamine | Plainfield |
| | | | NJ |
| PF-5 SI01-2 TiO2 CR-50 | titanium dioxide | C9-15 | Kobo |
| | | Fluoroalcohol | Products, |
| | | Phosphates (And) | Inc. South |
| | | Methicone (And) | Plainfield |
| | | Alumina | NJ |
| FC2-55 Black BL-100 | Iron Oxide | C9-15 | Kobo |
| | | Fluoroalcohol | Products, |
| | | Phosphate (And) | Inc. South |
| | | Microcrystalline | Plainfield |
| | | Cellulose | NJ |
| BGRO-BAS2 | Iron Oxide | Triethoxysilylethyl | Kobo |
| | | Polydimethylsiloxy | Products, |
| | | ethyl Hexyl | Inc. South |
| | | Dimethicone | Plainfield |
| | | | NJ |
| BGRO-TTS2 | Iron Oxide | Isopropyl Titanium | Kobo |
| | | Triisostearate/ | Products, |
| | | Triethoxycaprylylsilane | Inc. South |
| | | Crosspolymer | Plainfield |
| | | | NJ |
| BGBO-TTM2 | Iron Oxide | Isopropyl titanium | Kobo |
| | | triisostearate/Methicone | Products, |
| | | Crosspolymer | Inc. South |
| | | | Plainfield |
| | | | NJ |
| PF-5 BLACK BL-100 | Iron Oxide | C9-15 | Kobo |
| | | Fluoroalcohol | Products, |
| | | Phosphates | Inc. South |
| | | | Plainfield |
| | | | NJ |
| AS-5123 alkyl silane treated | Iron Oxide | Triethoxycaprylylsilane | Color |
| red iron oxide | | | Techniques, |
| | | | Inc., South |
| | | | Plainfield |
| | | | NJ |
| DI-9825 dimethicon treated | Titanium Dioxide | dimethicone | Color |
| Micro TiO2 | | | Techniques, |
| | | | Inc., South |
| | | | Plainfield |
| | | | NJ |
| PFD-8146 Fluoropropyl | Iron Oxide | Fluoropropyl | Color |
| Methicone treated black | | Methicone | Techniques, |
| oxide | | | Inc., South |
| | | | Plainfield |
| | | | NJ |
| DL-9146 Hydrophobic Black | Iron Oxide | methicone | Color |
| Iron Oxide | | | Techniques, |
| | | | Inc., South |
| | | | Plainfield |
| | | | NJ |
| MM-4146 Magnesium | Iron Oxide | Magnesium | Color |
| Myristate/I.O. Black | | Myristate | Techniques, |
| | | | Inc., South |
| | | | Plainfield |
| | | | NJ |
| Oleosperse RIO | Iron Oxide | Dimethicone | Presperse, |
| | | | Inc. |
| | | | Somerset, |
| | | | NJ |
| Lecithin (LT/VLI) Treated | Iron Oxide | Hydrogenated | U.S. |
| Iron Oxide | | Lecithin | Cosmetics |
| | | | Corporation, |
| | | | Dayville |
| | | | CT |
| Lipoamino Acid Treated Iron | Iron Oxide | Palmitoyl Proline/ | U.S. |
| Oxide | | Magnesium | Cosmetics |
| | | Palmitoyl | Corporation, |
| | | Glutamate/Sodium | Dayville |
| | | Palmitoyl | CT |
| | | Sarcosinate/ | |
| | | Palmitic Acid | |
| Metal Soap (MT/MPT) Treated | Iron Oxide | Aluminum | U.S. |
| Iron Oxide | | Dimyristate | Cosmetics |
| | | | Corporation, |
| | | | Dayville |
| | | | CT |
| Dimethicone (SAT) Treated | Iron Oxide | Dimethicone | U.S. |
| Iron Oxide | | | Cosmetics |
| | | | Corporation, |
| | | | Dayville |
| | | | CT |
| Dimethicone/Glyceryl | Iron Oxide | Dimethicone/Glyceryl | U.S. |
| Rosinate (ST-PEG/MOD) | | Rosinate | Cosmetics |
| Treated Iron Oxide | | | Corporation, |
| | | | Dayville |
| | | | CT |

Shouldn't we cover organic pigments such as (taken from Epson's U.S. Pat. No. 7,030,0174):

Examples of the color pigment used for yellow ink include C.I. Pigment Yellow 1 (Fast Yellow G), 2, 3, 12 (Disazo Yellow AAA), 13, 14, 16, 17, 24, 34, 35, 37, 42 (yellow iron oxide), 53, 55, 73, 74, 75, 81, 83 (Disazo Yellow HR), 93, 95, 97, 98, 100, 101, 104, 108, 109, 110, 114, 117, 120, 128, 129, 138, 151, 153 and 154.

Examples of the color pigment used for magenta ink include C.I. Pigment Red 1, 2, 3, 5, 7, 12, 17, 22 (Brilliant Fast Scarlet), 23, 31, 38, 48(Ca), 48(Mn), 48:2 (Permanent Red 2B (Ba)), 48:2 (Permanent Red 2B (Ca)), 48:3 (Permanent Red 2B (Sr)), 48:4 (Permanent Red 2B (Mn)), 49:1, 52:2, 53:1. 57 (Ca), 57:1 (Brilliant Carmine 6B), 60:1, 63:1, 63:2, 64:1, 81 (Rhodamine 6G Lake), 83, 88, 101 (iron oxide red), 104, 105, 106, 108 (Cadmium Red), 112, 114, 122 (Quinacridone Magenta), 123, 146, 149, 166, 168, 170, 172, 177, 178, 179, 184, 185, 190, 193, 202, 209 and 219.

Examples of the color pigment used for cyan ink include C.I. Pigment Blue 1, 2, 3, 15 (Phthalocyanine Blue R), 15:1, 15:2, 15:3 (Phthalocyanine Blue G), 15:4, 15:6 (Phthalocyanine Blue E), 15:34, 16, 17:1, 22, 56, 60 and 63, and C.I. Vat Blue 4 and C.I. Vat Blue 60.

Examples of the color pigment used for green ink include C.I. Pigment Green 1, 4, 7, 8, 10, 17, 18 and 36.

The colored particulate benefit agents useful in the compositions and methods of this invention can be spherical, spheroid, hemispherical, planar, flakes, or irregular in shape. Non-pigment particles can be made of polymethyl methacrylates, polyethylene, ethylene/acrylates copolymer, cellulose, nylon, polyurethane, silicone resin, mica, talc, sericite, or silica, for example. The particles can be inherently colored, or they can be mixed with colorants, such as dyes, pigments, or lakes, to give them color. Alternatively, they can be surface treated with colorants, such as dyes or pigments. Nonlimiting examples of colored particles include a 50/50 mixture of polymethyl methacrylate beads and red iron oxide pigments, and mixtures of titanium dioxide, iron oxide, and micas (available from LCW Sensient Technologies as the Covapearl® AS line).

The anionic pigments and particles of this invention preferably have an Isoelectric Point of about 7 to about 2, and a zeta potential of less than about −20 mV.

The anionic pigments and particles used in this invention have a concentration range of from about 0.05% to about 10% by weight, more preferably from about 0.1% to about 5% by weight, and even more preferably from about 0.5% to about 2% by weight.

### Anionic Dispersants

Anionic coated pigments and particles can be used in the second composition of this invention without the need for an anionic dispersant. This is due to the fact that their anionic surface charge renders them dispersible in the composition and able to be deposited evenly onto the hair surface after the hair surface is treated with the cationic compound of the first cosmetic composition. Other pigments and particles, including those that are uncoated as well as those with hydrophilic and hydrophobic coatings, require the presence of an anionic dispersant to disperse them in the second composition, maintain the stability of the composition, and enable even deposition of the pigments or particles on the treated substrate.

Examples of anionic dispersants include, without limitation, acrylates, sulfates, sulfonates, sulfosuccinates, phosphates, phosphonates, and the like. Specific examples of anionic dispersants suitable for use in the compositions and methods of this invention include anionic surfactants, such as sodium laureth sulfate, sodium dioctylsulfosuccinate, sodium methyl oleoyl taurate, laureth-1 phosphate, linear alcohol ethyoxy phosphate; anionic polymers, such as polyacrylate sodium salt, carbopol, xanthan gum, acrylic acid/vinyl ester copolymer, synthetic anionic polymers include sodium laureth sulfate (SLES), sodium polystyrene sulfonate, sodium polymethacrylate, sodium polynapthalenesulphonate, acrylates/C10-30 alkyl acrylate crosspolymer, acrylates/beheneth-25 methacrylate copolymer, acrylates/steareth-20 methacrylate copolymer, acrylates/VA crosspolymer, acrylic acid/acrylonitrogens copolymer, carbomerPVM/MA decadiene crosspolymer, acrylates copolymer, octylacrylamide/acrylates/butylaminoethylmethacrylate copolymer, PVM/MA copolymer, VA/crotonates/vinyl neodecanoate copolymer, glyceryl polymethacrylate, Aculyn® polymers available from Rohm and Haas Company in Spring House, Pa., and Structure® XL, available from National Starch and Chemical Company in Bridgewater, N.J.; and mixtures of these.

### Compositions

The compositions of this invention may be prepared in the form of formulations known to be useful for cosmetic skin and hair products. For example, they can be in the form of shampoos, conditioners, lotions, rinses, dispersions, emulsions, gels, cream gels, creams, pastes, sticks, suspensions, sprays, mousse, aerosols or foams. To the compositions of the invention may be added other substances, auxiliary agents, for example those commonly used for cosmetic products in general. Such materials include, for example, thickeners (for example clays, starches, polyacrylic acid and the derivatives thereof), cellulose derivatives, lanolin derivatives, vitamins or provitamins, (for example biotin, vitamin C, tocopherols or D-panthenol), antigrease agents, inorganic or organic acids (for example lactic acid, citric acid, glycolic acid or phosphoric acid), preservatives (for example para-hydroxybenzoate esters), nonaqueous solvents, antioxidants (for example tocopherols or the esters thereof), dyes and fragrances or perfumes, UV light-absorbing inorganic particles and others known to those of ordinary skill in the art.

### Other Cosmetic Components and Additives

In addition to the above-described ingredients, other common cosmetic components and additives may be incorporated in the compositions of this invention, as long as the basic properties of the compositions and the ability to color or impart other benefits to keratin-containing substrates are preserved. Such ingredients include, but are not limited to, humectants, emollients, moisturizers, inorganic salts, fragrances, hydrotropes, foam stabilizers, preservatives, water softening agents, acids, bases, buffers and the like. Optional components may be present in weight percentages of less than about 2% each, and from about 5% to about 10% by weight of the composition in total.

### Cosmetically Acceptable Carriers:

The compositions of this invention preferably contain one or more cosmetically-acceptable carriers. Preferably, such carriers include water. Organic solvents may also be included in order to facilitate manufacturing of the composition or to provide esthetic properties, such as viscosity control. Suitable solvents include the lower alcohols (i.e., C2-C6 alcohols), such as ethanol, propanol, isopropanol, butanols, pentanols, and hexanols; glycol ethers, such as 2-butoxyethanol, ethylene glycol monoethyl ether, propylene glycol and diethylene glycol monoethyl ether or monomethyl ether; and the mixtures thereof. A preferred organic solvent in this invention is ethanol.

Non-aqueous solvents may be present in the compositions of the present invention in an amount of about 1% to about 50%, and in particular about 5% to about 25%, by weight of the total weight of the carrier in the composition.

The compositions of this invention should be stable to phase or ingredient separation at a temperature of about 25° C. for an indefinite period of time, or at least for 5 weeks at a temperature of 45° C. Thus, the compositions of this invention have demonstrated sufficient stability to phase and ingredient separation at temperatures normally found in commercial product storage and shipping to remain unaffected for periods of at least one year.

The compositions of this invention may be utilized in any types of products that impart color to keratin-containing substrates, including, but not limited to the following: hair color, powders, make-up, mascara, foundations, lip color, blush, cosmetic pencils, sunless tanning products, wool fabric coloring, tooth whitening products, nail color, and the like.

Keratin-containing substrates to which the compositions and methods of this invention may be applied and to which color or other benefits may be imparted include hair, skin, teeth, nails, wool, fur, and the like.

Although Examples 2 and 3 set forth below recite methods of and compositions for coloring hair, the method described herein may be applied to other keratin-containing substrates that are amenable to treatment with particulate benefit agents according to the methods of this invention. Treating the hair with the compositions of this invention is generally carried out by: (1) applying to dry or wet hair an effective amount of the first cosmetic composition of the invention; (2) distributing the first composition of this invention more or less evenly throughout the hair such that it contacts substantially all the hair or other substrate which is intended to be colored or otherwise affected by particulate benefit agent. This permits the cationic compound of the first compositions of this invention to be applied thoroughly and evenly throughout the hair or other substrate to form a layer on the hair. This step may be accomplished by rubbing the first composition throughout the hair manually or using a hair appliance such as a comb for up to about 20 minutes; and (3) optionally, rinsing said hair or other substrate with water so as to remove excess material that has not deposited onto the hair; (4) applying to dry or wet hair an effective amount of the second cosmetic composition of the invention; (5) distributing the second composition of this invention more or less evenly throughout the hair such that it contacts all the hair or other substrate which is intended to be colored or otherwise affected by the particulate benefit agent. This permits the particulate benefit agent of the second cosmetic composition of this invention to be applied thoroughly and evenly throughout the hair or other substrate. This step may be accomplished by rubbing the second cosmetic composition throughout the hair manually or using a hair appliance such as a comb for up to about 20 minutes or as long as is sufficient to expose substantially all of the hair or other substrate to said second cosmetic composition; and (6) optionally, rinsing said hair or other substrate with water so as to remove excess material that has not deposited onto the hair. Treating the hair with the compositions of the invention may be carried out by applying rinse-off types of compositions, or by applying leave-on types of compositions, such as hair spray, cream, or solution, directly to hair without rinsing the hair.

The compositions and methods of this invention are further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Colorimetric Measurement

Color deposition on hair tresses, wool swatches, and other keratin-containing substrates may be measured using an UltraScan PRO spectrophotometer, available from Hunter Associates Laboratory, Inc., of Reston, Va. In general, untreated hair swatches and hair swatches treated with various compositions are placed on a colorimeter for the quantitative evaluation of color. The Hunter L*, a*, and b* values are determined for both the treated and untreated hair swatches, and the total color change between treatments (ΔE) is determined.

The measurement is made by placing a hair tress into the channel of a hair tress holding port fitted to the colorimeter. Colorimetric measurements (Hunter L*, a*, and b*) of the hair tress are made at 0.5-inch intervals from the root end to the tip end until a total of five measurements per tress are completed. The five measurements are then averaged for the tress.

The Hunter L* measurement refers to the lightness axis, where more positive numbers are lighter and more negative numbers are darker. The Hunter a* value refers to the red-green axis, where more positive numbers are more red and more negative numbers are more green. The Hunter b* value refers to the blue-yellow axis, where more positive numbers are more blue and more negative numbers are more yellow.

The color uptake onto a substrate is determined by subtracting the value of the original tress from the value of the treated one. The color loss, for example after washing, is determined by subtracting the value of the treated tress from the value of the post-washed tress. An equation for percentage color loss is shown by the equation:

Color loss(%)=ΔX(Loss)/ΔX(Uptake)×100

where
ΔX(Uptake)=X(treated)−X(original);
ΔX(Loss)=X(post-wash)−X(treated); and
X=L*, a*, or b*

The total color change, or ΔE, representing the total color change between treatments, is given by the equation:

ΔE=\sqrt{└(L₂*−L₁*)²+(a₂*−a₁*)²+(b₂*−b₁*)²┘}

where the subscripts 1 and 2 represent original, treated, and/or post-washed values.

Color uniformity is determined from the standard deviation of multiple measurements from root to tip within a single tress. The smaller the standard deviation, the more uniform the color on the tress is.

### Example 1

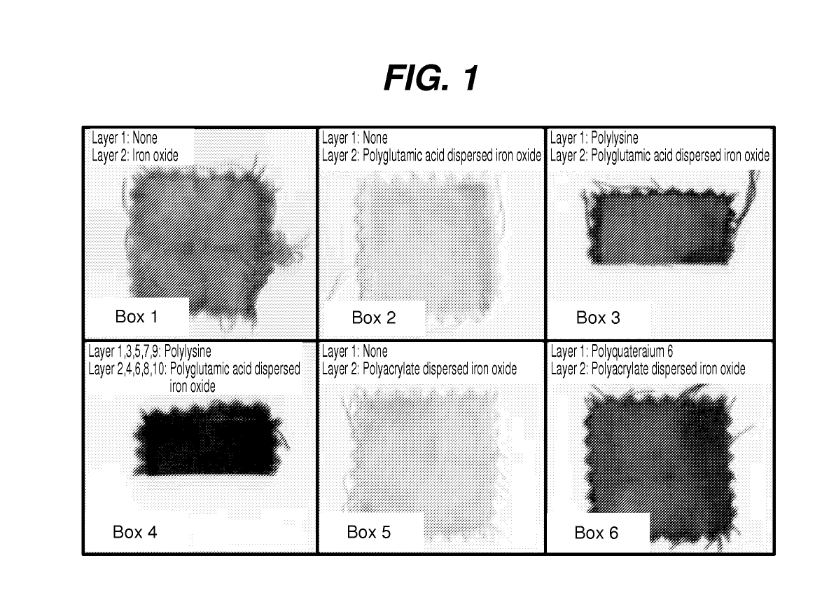

Example 1 demonstrates the deposition of colored pigments on wool swatches according to the compositions and methods of this invention. Swatches of white wool fabric available from Testfabrics, Inc., as Style 541 Worsted Gabardine, Lot 5675 were treated with iron oxide under various conditions according to Table 6. Some of the swatches were also first treated with a composition containing a cationic compound according to the first composition of this invention, and, as a comparison, others were not. Photographs of the swatches are shown in FIG. 1.

TABLE 6

| | FIG. 1 | | |
|---|---|---|---|
| Sample | reference | First composition | Second composition |
| 1A | Box 1 | None | Iron oxide |
| 1B | Box 2 | None | Polyglutamic acid dispersed |
| | | | iron oxide |
| 1C | Box 3 | Polylysine | Polyglutamic acid dispersed |
| | | | iron oxide |
| 1D | Box 4 | Polylysine X5 | Polyglutamic acid dispersed |
| | | | iron oxide X5 |
| 1E | Box 5 | None | Polyacrylate dispersed iron |
| | | | oxide |
| 1F | Box 6 | Polyquaternium 6 | Polyacrylate dispersed iron |
| | | | oxide |

All first compositions used in Example 1 contained 1 wt % of the cationic compound in DI water. Wool swatches were immersed into 20 mL of the cationic solution. The second composition of Sample 1A contained 0.5 wt % of iron oxide (Kobo Black Iron Oxide 77499) in DI water. The second composition of Samples 1B-1F contained 5 wt % iron oxide (Kobo Black Iron Oxide 77499) in DI water plus 0.5 wt % of the specified anionic polypeptide or polymer.

Referring now to FIG. 1, it can be seen in Box 1 that treatment with iron oxide pigment alone results in some blotchy, or nonuniform, deposition of the pigment on the wool. In Box 2, where the swatch was treated only with a composition in which iron oxide was dispersed with polyglutamic acid, there was almost no deposition of pigment. In Box 3, when the swatch was treated with a first composition containing polylysine and subsequently treated with a second composition containing iron oxide dispersed with polyglutamic acid, the deposition of pigment was heavier and more uniform than with either of Samples 1A or 1B, the swatches where no first treatment was made. Box 4 shows the effects of treating the swatch of Sample 1D with multiple sequential treatments of polylysine followed by polyglutamic acid dispersed iron oxide. Sample 1D was treated five times with the first composition containing polylysine alternating with five treatments of the second composition containing polyglutamic acid/iron oxide. It can be seen that the intensity of the color for Sample 1D is much darker and more uniform than for the other samples. Box 5 shows the effect of treating the swatch with only iron oxide dispersed with polyacrylate, i.e., without a first treatment with a cationic compound. The swatch in Box 5 shows very little color acquisition. Box 6 shows the effect of treating the swatch first with a composition containing polyquaternium 6 followed by treatment with a second composition containing iron oxide dispersed with polyacrylate. Like the swatch shown in Box 3, the swatch shown in Box 6 shows heavier, more uniform deposition of pigment than the swatches in Boxes 2 and 5, which did not receive treatment with a cationic compound.

### Example 2

Cationic peptides and proteins were assessed for iron oxide deposition and retention using the method described below. All of the cationic peptides and proteins were obtained from Sigma Aldrich of St. Louis, Mo.

The human hair used in this example was natural white hair in 250 mg tress samples. Such hair is available commercially, for example from International Hair Importers and Products (Bellerose, N.Y.), and is also available in different colors, such as brown, black, red, and blonde, and in various types, such as African-American, Caucasian, and Asian.

All colorimetric analyses were conducted using a Konic-Minolta colorimeter. The untreated hair tress samples used in this example were each analyzed colorimetrically before any treatment to obtain an untreated colorimetric measurement (Hunter L*, a*, b*).

Each cationic peptide or protein was formulated into a first treatment composition by weighing 0.05 mg of the peptide or protein into a scintillation vial to which 2 ml of 25 mM tris buffer solution was then added. The solution was mildly mixed by tumbling (end over end mixing) at 50 rpm for 5 minutes. Two milliliters of this solution was applied to 250 mg of swatched hair in the samples of this example.

The second treatment composition was made by first measuring 2 mg of red iron oxide (UNIPURE LC381EM, Sensient Technologies) into a scintillation vial. A 20% dispersion was first made by adding the 2 mg of iron oxide to 8 ml of 25 mM tris buffer solution. This sample was then homogenized with an ULTRA TURAX T25 basic homogenizer at 24,0000 rpm for 5 minutes. The homogenization step was repeated 2-3 times to ensure a stable dispersion of pigment. This dispersion was then diluted to 0.25% pigment with 25 mM tris buffer solution. One milliliter of this 0.25% dispersion was then applied to 250 mg of swatched hair in the samples of this example.

The above-specified amounts of first and second treatment compositions were placed in 50 ml plastic hexagonal weighing boats.

A tress of untreated hair, measuring 0.5×4 cm, was placed into weighing boat with the first treatment composition with mild finger embrocation for 1 minute. The hair sample was then allowed to stand in the first treatment composition for 9 minutes for a total treatment time of 10 minutes. The tress was then removed and rinsed with 95-98° F. tap water for 15 seconds per side. While the tress was still wet, it was placed into the weighing boat with the second composition containing the pigment, subjected to mild finger embrocation for 1 minute, and allowed to stand for 9 minutes for a total treatment time of 10 minutes. The tress was then removed, rinsed under running 95-98° F. tap water for 30 seconds, and allowed to air dry.

Each tress was then further analyzed colorimetrically to determine the color uptake.

Table 7 shows the cationic peptides and proteins that were used in this example and their color uptake values (ΔE).

TABLE 7

| | Sigma | | | Color |
|---|---|---|---|---|
| Sample # | Aldrich # | Cationic peptide/protein | CAS Number | uptake (ΔE) |
| 2A | P7658 | Poly (D-Glu, D-Lys) | 119039-80-4 | 26 |
| | | hydrobromide D-Glu:D-Lys | | |
| | | (6:4), mol wt 20,000-50,000 | | |
| 2B | P1152 | Poly (Ala, Glu, Lys, Tyr) | 28704-27-0 | 28 |
| | | 6:2:5:1 hydrobromide | | |
| | | Ala:Glu:Lys:Tyr (6:2:5:1), mol | | |
| | | wt 20,000-30,000 | | |
| 2C | P9306 | Poly(Arg, Pro, Thr) | 133552-00-8 | 8 |
| | | hydrochloride Arg:Pro:Thr | | |
| | | (1:1:1), mol wt 5,000-20,000 | | |
| 2D | P9431 | Poly(Arg, Pro, Thr) | 133552-00-8 | 16 |
| | | hydrochloride Arg:Pro:Thr | | |
| | | (6:3:1), mol wt 10,000-30,000 | | |
| 2E | P0411 | Poly(Arg, Trp) hydrochloride | 119039-92-8 | 11 |
| | | Arg:Trp (4:1), mol wt 20,000-50,000 | | |
| 2F | P0650 | Poly(Glu, Lys) hydrobromide | 119039-90-6 | 5 |
| | | Glu:Lys (1:4), mol wt 150,000-300,000 | | |
| 2G | P4409 | Poly(Glu, Lys, Tyr) sodium | 118539-64-3 | 20 |
| | | salt Glu:Lys:Tyr (6:3:1), mol | | |
| | | wt 20,000-50,000 | | |
| 2H | 82357 | Poly(Glu, Tyr) sodium salt | 97105-00-5 | 3 |
| | | BioChemika, mol wt 20,000-50,000, | | |
| | | Glu:Tyr 4:1 | | |
| 2J | P0151 | Poly(Glu, Tyr) sodium salt | 97105-00-5 | 5 |
| | | Glu:Tyr (1:1), mol wt 20,000-50,000 | | |
| 2K | P3150 | Poly(Lys, Phe) 1:1 | 26700-39-0 | 10 |
| | | hydrobromide mol wt 20,000-50,000 | | |
| 2L | P9285 | Poly(Lys, Trp) 4:1 | 119039-85-9 | 29 |
| | | hydrobromide mol wt 20,000-50,000 | | |
| 2M | P4659 | Poly(Lys, Tyr) hydrobromide | 41705-04-8 | 23 |
| | | Lys:Tyr (4:1), mol wt 20,000-50,000 | | |

It can be seen from the data in Table 7 that, although pigment deposition, or uptake, was demonstrated with all of the cationic peptides and proteins used here, certain of the samples, that is, 2A, 2B, 2G, 2L, and 2M, had higher pigment deposition. All of these samples contained lysine.

### Example 3

The five cationic peptides and proteins demonstrating the highest pigment deposition from Example 2 were further tested for color retention after multiple shampoos. The samples were prepared as in Example 2 above, except that 0.025 mg of each cationic peptide or protein was used in the first composition of each sample. The second treatment composition and the sequential application procedure were the same as described in Example 2.

Color Retention after Shampoo

About 12-24 hours after pigmenting the tresses, they were assessed for retention of color after shampooing. A 2% SLES solution was prepared by diluting RHODAPEX ES-2K sodium laureth sulfate (26%), available from Rhodia, Cranbury, N.J., in deionized water. Forty (40) grams of this solution was measured into a 4 ounce jar. The pigmented tress was added to the jar, and the jar was placed on an orbital shaker at 200 rpm for 5 minutes. The tress was then rinsed under running lukewarm tap water for about 30 seconds and air-dried at room temperature.

Each tress was then analyzed colorimetrically to determine color retained. This was reported as “Color retention”. This procedure was then repeated for determining color retention after multiple shampoos.

Table 8 shows the initial color uptake and the color retention after first and second shampoos when the first composition contained the better performing cationic proteins and peptides from Table 7.

TABLE 8

| Sample | Sigma | | Initial | 1 Shampoos | 2 Shampoos |
|---|---|---|---|---|---|
| # | Aldrich # | CAS Number | ΔE | ΔE | ΔE |
| 2A | P7658 | 119039-80-4 | 19 | 15 | 11 |
| 2B | P1152 | 28704-27-0 | 13 | 10 | 8 |
| 2C | P4409 | 118539-64-3 | 15 | 12 | 9 |
| 2D | P9285 | 119039-85-9 | 17 | 11 | 10 |
| 2E | P4659 | 41705-04-8 | 19 | 13 | 10 |

It can be seen from the data in Table 8 that, although some of the color is washed out by the repeated shampooings, color is still retained after two shampoos.

### Example 4

Cationic polymers were assessed for iron oxide deposition and retention using the methods described in Examples 2 and 3 except that 0.025 mg cationic polymer was substituted for the cationic peptide/protein in the first treatment composition. The UCARET™ and SoftCAT™ cationic polymers in this example were cellulose polymers obtained from Amerchol Corporation of Piscataway, N.J. Additional cationic polymers, POLYSURF 67CS (available from Hercules Aqualon Division in Wilmington, Del.) and JAGUAR C-17 (available from Rhodia, Cranbury, N.J.), and nonionic polymers, KLUCEL MCS (Hercules Aqualon Division in Wilmington, Del.), NATROSOL 250HHR (Hercules Aqualon Division in Wilmington, Del.), and JAGUAR HP-8 (Rhodia, Cranbury, N.J.), were also assessed for deposition and retention according to the same method. Table 9 shows the polymers that were used and their initial color uptake, as well as their color retention after first and second shampoos.

TABLE 9

| | | | Initial | 1 Shampoo | 2 Shampoos |
|---|---|---|---|---|---|
| Sample # | Polymer | Charge | (ΔE) | (ΔE) | (ΔE) |
| 4A | SoftCAT ™ SK-M | Cationic | 38 | 35 | 31 |
| 4B | SoftCAT ™ SK-MH | Cationic | 38 | 31 | 28 |
| 4C | SoftCAT ™ SK-H | Cationic | 38 | 34 | 30 |
| 4D | SoftCAT ™ SL 5 | Cationic | 36 | 33 | 29 |
| 4E | SoftCAT ™ SL 30 | Cationic | 38 | 35 | 30 |
| 4F | SoftCAT ™ SL 60 | Cationic | 38 | 31 | 29 |
| 4G | UCARE ™ Polymer JR 400 | Cationic | 39 | 34 | 30 |
| 4H | UCARE ™ Polymer JR 30M | Cationic | 37 | 34 | 30 |
| 4K | UCARE ™ Polymer LR 400 | Cationic | 36 | 31 | 25 |
| 4L | UCARE ™ Polymer LR 30M | Cationic | 37 | 32 | 28 |
| 4M | UCARE ™ Polymer JR 125 | Cationic | 31 | 27 | 23 |
| 4N | SoftCAT ™ SX-400H | Cationic | 37 | 35 | 29 |
| 4P | SoftCAT ™ SX-1300H | Cationic | 38 | 34 | 28 |
| 4Q | UCARE ™ Polymer LK | Cationic | 36 | 32 | 28 |
| 4R | JAGUAR C-17 | Cationic | 37 | 32 | 29 |
| 4S | POLYSURF 67-CS | Cationic | 35 | 30 | 27 |
| 4T | KLUCEL MCS | Nonionic | 30 | 26 | 19 |
| | | (comparative) | | | |
| 4U | NATROSOL 250HHR | Nonionic | 25 | 19 | 16 |
| | | (comparative) | | | |
| 4V | JAGUAR HP-8 | Nonionic | 13 | 10 | 8 |
| | | (comparative) | | | |

It can be seen that all of the cationic polymers in this example demonstrated good color uptake and good retention after two shampooings. Additionally, the nonionic polymers exhibited less pigment deposition than the cationic polymers, both initially and after the shampooings.

### Example 5

The cationic proteins and peptides of Example 3 and the cationic polymers of Example 4, as well as the comparative nonionic polymers of Example 4, were assessed for durability. In this example, durability is a measure of how much pigment is deposited onto the layer produced by treatment with the cationic first compound when the hair is shampooed between treatment with the first composition and treatment of the second composition. Higher ΔE values indicate that the cationic layer is being retained on the hair even after shampooing, as evidenced by colorimetric measurement of pigment deposition after the shampooing. The procedure used to measure durability is described below.

### Durability

The tresses were assessed for retention of the cationic compound after treatment with the first composition and then shampooing. These shampooed tresses were then treated with the second composition containing the pigment and analyzed colorimetrically.

The shampooing was conducted by clipping 5-10 of the cationic-compound treated tresses to a clipboard, and wetting all the tresses with tap water. Shampoo was then applied by evenly applying to each tress a 1 ml aliquot of 2% SLES solution by Eppendorf pipettor. After all the tresses had shampoo on them, each tress was brushed 10 times with an eight-inch horse hair brush, rinsed under lukewarm tap water, and brushed an additional 10 times. The tresses were then flipped over and the shampooing, brushing, and rinsing steps were repeated.

These shampooed tresses were then treated with the second composition containing the pigment as described in Example 2 above and analyzed colorimetrically to determine the color uptake. This was reported as “Durability.”

The results of the durability testing for the proteins, peptides, and polymers of Examples 3 and 4 are shown in Table 10.

TABLE 10

| | |
|---|---|
| First composition | Durability (ΔE) |

Cationic polymers

| | | |
|---|---|---|
| Polyquaternium-10 | UCARE LK | 43 |
| | UCARE LR-400 | 45 |
| | UCARE JR-125 | 41 |
| | UCARE JR-400 | 42 |
| | UCARE LR-30M | 44 |
| | UCARE JR-30M | 44 |
| Polyquaternium-67 | SoftCAT SK-H | 45 |
| | SoftCAT SK-M | 47 |
| | SoftCAT SK-MH | 46 |
| | SoftCAT SX-400H | 39 |
| | SoftCAT SX-1300H | 41 |
| Guar Hydroxypropyl | JAGUAR C-17 | 42 |
| trimonium chloride | | |
| | POLYSURF 67CS | 20 |
| Polyquaternium-67 | SoftCAT SL-5 | 46 |
| | SoftCAT SL-30 | 46 |
| | SoftCAT SL-60 | 48 |

Cationic peptides and proteins

| | |
|---|---|
| P4659 | 45 |
| P7658 | 19 |
| P9285 | 44 |
| P1152 | 41 |
| P4409 | 17 |

Nonionic polymers (comparative)

| | | |
|---|---|---|
| Hydroxypropylcellulose | KLUCEL MCS | 6 |
| Hydroxypropylguar | JAGUAR HP-8 | 8 |
| Hydroxyethylcellulose | NATROSOL 250HHR | 10 |

It can be seen that all of the cationic peptides, proteins, and polymers demonstrate better durability than the nonionic polymers.

What is claimed is:

1. A method for imparting a particulate benefit agent to a keratin-containing substrate comprising the following steps sequentially:
a) providing a first cosmetic composition comprising at least one cationic compound selected from the group consisting of cationic proteins, cationic peptides, cationic polymers, and the mixtures thereof;
b) applying said first cosmetic composition to the keratin-containing substrate for a time period sufficient for at least one said cationic compound to be deposited on the substrate and form a layer;
c) providing a second cosmetic composition comprising at least one anionic pigment; and
d) applying said second cosmetic composition to the keratin-containing substrate for a time period sufficient for at least one said anionic pigment to be deposited on said layer.

2. A method according to claim 1, wherein said cationic compound is a cationic protein.

3. A method according to claim 2, wherein said cationic protein is selected from the group consisting of lysozyme, avidin, antimicrobial Proteins, RNA or DNA binding proteins, proteases, methylated collagen, Cytochrome C, proteins involved in the aging process, Platelet Factor 4, protamine sulfate and mixtures thereof.

4. A method according to claim 3 wherein said antimicrobial proteins are selected from the group consisting of: magainin, defensins, cathelicidin and mixtures thereof.

5. A method according to claim 3 wherein said RNA or DNA binding proteins are selected from the group consisting of histones, ribonuclease A, Deoxyribonuclease and mixtures thereof.

6. A method according to claim 3 wherein said proteases are selected from the group consisting of Trypsin, Chymotrypsin, Papain, Caspase and mixtures thereof.

7. A method according to claim 3 wherein said protein involved in the aging process is +Telomerase.

8. A method according to claim 1, wherein said cationic compound is a cationic peptide.

9. A method according to claim 8, wherein said cationic peptide is selected from the group consisting of polylysine, polyarginine, polyhistidine, copolymers and peptides containing a greater number of basic amino acids than acidic amino acids, and the mixtures thereof.

10. A method according to claim 1, wherein said cationic compound is a cationic polymer.

11. A method according to claim 10, wherein said cationic polymer is a naturally-occurring cationic polymer selected from the group consisting of chitosan, polyquaternium-4, polyquaternium-10, polyquaternium-24, polyquaternium-67, and the modifications thereof.

12. A method according to claim 10, wherein said cationic polymer is a synthetic cationic polymer selected from the group consisting of synthetic cationic polymers comprising one or more primary amines, synthetic cationic polymers comprising one or more secondary amines, synthetic cationic polymers comprising one or more tertiary amines, synthetic cationic polymers comprising one or more quaternary amines, and the mixtures thereof.

13. A method of according to claim 12, wherein said synthetic cationic polymer is selected from the group consisting of poly methacrylamidopropyltrimethylammonium chloride, polyquaternium-1, polyquaternium-2, polyquaternium-5, polyquaternium-6, polyquatenium-7, polyquaternium-8, polyquaternium-11, polyquaternium-16, polyquaternium-17, polyquaternium-18, polyquaternium-22, polyquaternium-27, polyquaternium-28, polyquaternium 31, polyquaternium-39, polyquaternium-43, polyquaternium-44, polyquaternium-46, polyquaaternium-47, polyquaternium-53, polyquaternium-55, PVP/dimethylaminoethyl methacrylate copolymer, VP/dimethylaminoethyl methacrylate copolymer, VP/DMAPA acrylate copolymer, VP/vinyl caprolactam/DMAPA acrylates copolymer, vinylcaprolactam/PVP/dimethylaminoethylmethacrylate copolymer, and the mixtures thereof.

14. A method according to claim 1, wherein said anionic pigment is selected from the group consisting of titanium oxides, zinc oxides, aluminum oxides, iron oxides, manganese oxides, silicon oxides, silicates, cerium oxide, zirconium oxides, barium sulfate and mixtures thereof.

15. A method according to claim 1, wherein said anionic pigment is an anionic microparticle.

16. A method according to claim 15, wherein said anionic microparticle has a diameter of from about 1 to about 1000 micrometers.

17. A method according to claim 1, wherein said anionic pigment is an anionic nanoparticle.

18. A method according to claim 17, wherein said anionic nanoparticle has a diameter of from about 1 to about 1000 nanometers.

19. A method according to claim 1, wherein said keratin-containing substrate is selected from the group consisting of hair, skin, nails, teeth, tissues, wool and fur.

20. A method according to claim 1, wherein said cationic compound has an Isoelectric Point of from about 8 to about 12.

21. A method according to claim 1, wherein said anionic pigment has an Isoelectric Point of from about 7 to about 2.

22. A method according to claim 1 wherein the zeta potential of the anionic pigment is sufficient to maintain said composition as a stable dispersion.

23. A method according to claim 22 wherein the zeta potential of the anionic pigment is less than about −20 mV.

24. A method according to claim 1, wherein said cationic compound has a concentration of from about 0.000001% to about 10% by weight.

25. A method according to claim 24, wherein said cationic compound has a concentration of from about 0.001% to about 5% by weight.

26. A method of claim according to claim 25, wherein said cationic compound has a concentration of from about 0.01% to about 2% by weight.

27. A method according to claim 1, wherein said anionic pigment has a concentration of from about 0.05% to about 10% by weight.

28. A method according to claim 27, wherein said anionic pigment has a concentration of from about 0.1% to about 5% by weight.

29. A method of claim according to claim 28, wherein said anionic pigment has a concentration of from about 0.5% to about 2% by weight.

30. A cosmetic kit for coloring a keratin-containing substrate comprising:
a) a first container containing a first cosmetic composition comprising at least one cationic compound selected from the group consisting of cationic proteins, cationic peptides, cationic polymers, and the mixtures thereof;
wherein said first composition is applied to the keratin-containing substrate for a time period sufficient for at least one said cationic compound to be deposited on the substrate and form a layer, and rinsed off with water;
b) a second container containing a second cosmetic composition comprising at least one anionic pigment;
wherein said second cosmetic composition is applied to the keratin-containing substrate for a time period sufficient for at least one said anionic pigment to be deposited on said layer, and raised off with water.

31. A colored keratin-containing substrate prepared by the method of claim 1.
